(19) United States
(12) Patent Application Publication
(10) Pub. No.: US 2013/0327988 A1
(43) Pub. Date: Dec. 12, 2013

(54) PROCESS FOR THE PREPARATION OF IMIDES AND DERIVATIVES THEREOF AND USES

(71) Applicants: Roland Jacquot, Francheville (FR); Philippe Marion, Vernaison (FR)

(72) Inventors: Roland Jacquot, Francheville (FR); Philippe Marion, Vernaison (FR)

(73) Assignee: RHODIA OPERATIONS, Aubervilliers (FR)

(21) Appl. No.: 13/883,266

(22) PCT Filed: Oct. 31, 2011
(86) PCT No.: PCT/FR11/52548

§ 371 Date: May 2, 2013

(30) Foreign Application Priority Data
Nov. 2, 2010 (FR) 1058996

(51) Int. Cl. C07D 211/88; C09K 3/00

(52) U.S. Cl. CPC C07D 211/88; C09K 3/00
USPC 252/364; 546/243; 252/182.12

(57) ABSTRACT

A process for the preparation of imides and also the uses thereof, especially as intermediates for the preparation of solvents, in particular of diester solvents, is described. Further described is a process for preparing cyclic imides and derivatives thereof, especially the corresponding carboxylic acids.

The object of the present invention is a process for preparing imides as well as the uses thereof, notably as intermediates for preparing solvents, in particular diester solvents. More specifically, the invention relates to a process for preparing cyclic imides and derivatives thereof, notably the diesters of corresponding carboxylic acids.

Diesters are an interesting category of oxygenated solvents because of their technical performances and their low environmental impact. They have been substituted for chlorinated or oxygenated hydrocarbon solvents which are more aggressive for the environment in a significant number of industrial applications such as degreasing, stripping of paints . . . .

Today, several existing processes allow production of diesters.

A first preparation method, for example described in WO2007/141404, is a reaction between a dinitrile and a basic compound in a solvent, followed by acidification with a mineral acid in order to recover the corresponding carboxylic diacid which may then be esterified with an alcohol. The drawback of this method is notably that it generates salts, for example ammonium or sodium salts as co-products.

In order to overcome the problem of production of salts, a second method, notably described in WO2008/009792, was applied. This is a hydrolysis reaction in a vapor phase of a dinitrile, in the presence of an acid catalyst, leading to the formation of an imide, itself then transformed into a diester, by action of an alcohol in the presence of an acid catalyst.

A third preparation method, an alternative to the previous method, is notably described in WO2009/056477. It differs from the method described in WO2008/009792 by its second step in which the reaction between the imide and the alcohol may be achieved in basic catalysis or without a catalyst.

The first step for synthesis of the imide of both methods above has the drawback of forming ammonia, which is then recovered and treated.

Further, there always exists a need for improving the routes for accessing compounds such as diesters, by bringing value to products of fossil origin and/or by using bio-sourced raw materials. The need is located in rationalization and optimization of industrial tools.

One of the objects of the present invention is therefore to propose a process for manufacturing intermediates in the preparation of solvents, notably diesters, from compounds of fossil origin to which value should be provided and/or from bio-sourced compounds, not having the drawbacks of the methods of the prior art and notably not generating significant effluents or by-products possibly harmful for the environment.

The invention meets this need by proposing a process for preparing cyclic imide(s) by reaction between at least one carboxylic diacid and at least one dinitrile.

More specifically, the invention relates to a process for preparing cyclic imide(s) of the following formulae (I) and (II):

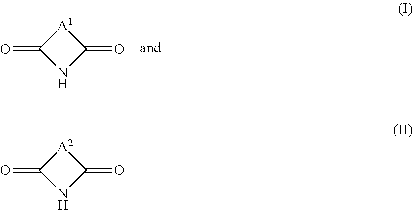

which comprises the reaction between at least one carboxylic diacid of the following formula (III):

HOOC-A¹-COOH (III)
and at least one dinitrile of the following formula (IV):

NC-A²-CN (IV)
formulae wherein A¹ and A² are identical or different and are selected from the following divalent hydrocarbon groups:
   an alkylene comprising a linear linked chain having 2 or 3 carbon atoms;
   an ortho-cycloalkylene having 5 or 6 carbon atoms;
   an alkenylene comprising a linear linked chain having 2 or 3 carbon atoms;
   an ortho-arylene having at least 6 carbon atoms,
one or more hydrogen atoms of said hydrocarbon groups may be substituted with a group R, R being selected from the following substituents: C₁-C₁₀ alkyl, C₅-C₆ cycloalkyl, C₆-C₁₀ aryl, C₆-C₁₀ alkylaryl, C₆-C₁₀ arylalkyl, hydroxy or halogeno; one or more hydrogen atoms of the alkylene and cycloalkylene groups may also be substituted with a group R′, R′ being a C1-C10 alkyledene.

When R is a C₁-C₁₀ alkyl group, it is preferably selected from linear or branched alkyls having from 1 to 4 carbon atoms. Advantageously, it is a methyl or an ethyl.

When R is a C₅-C₆ cycloalkyl group, it is selected from cyclopentyl and cyclohexyl.

When R is a C₆-C₁₀ aryl group, it is preferably a phenyl or a naphthyl.

When R is a C₆-C₁₀ alkylaryl group, it is preferably a benzyl.

When R is a C₆-C₁₀ arylalkyl group, a tolyl will preferably be selected.

When R′ is a C1-C₁₀ alkylidene group, it is preferably selected from the following groups: CH₂═, CH₃—CH═, (CH₃)₂C═, CH₃—CH₂—CH═, CH₃—CH₂—C(CH₃)═.

By <<halogeno>> is meant fluorine, chlorine, bromine and iodine.

By <<hydroxy>> is meant the —OH group.

As specific examples of an alkylene according to the invention, mention may be made of ethylene or propylene, either substituted or not with one or more, preferably only one, group R and/or R′ as defined above. More preferably, a selection will be made among the following alkylene groups: ethylene, propylene, ethyl-ethylene, 1-methyl-propylene and mixtures thereof.

As regards the ortho-cycloalkylene examples according to the invention, these are notably an ortho-cyclopentylene or an ortho-cyclohexylene, either substituted or not with one or several, preferably only one, group R as defined earlier. More preferably, a selection will be made among the following ortho-cycloalkylene groups: ortho-cyclopentylene, ortho-cyclohexylene and mixtures thereof.

The alkenylene groups may be suitable as hydrocarbon groups according to the invention and for example are —CH═CH— or —CH═CH—CH₂—, one or more of the hydrogens, preferably only one, may be substituted with a group R and/or R′ as defined earlier. More preferably, a selection will be made among the following alkenylene groups:
CH═CH—, —C(CH₃)═CH—, —C(CH₂CH₃)═CH—,
—CH═CH—CH₂—, —CH═CH—CH(CH₃)—, —CH═CH—CH(CH₂CH₃)—, —C(CH₃)═CH—CH₂—,
—CH═C(CH₃)—CH₂—, —CH═CH—C(═CH₂)— and mixtures thereof.

As specific examples of ortho-arylene according to the invention, mention will be made of ortho-phenylene either substituted or not with one or more, preferably only one, group R as defined earlier and later on in the description. More preferably, a selection will be made from ortho-phenylene, hydroxyl-ortho-phenylene and mixtures thereof.

The reaction scheme of the process of the invention is given hereafter in order to facilitate understanding of the invention without however limiting the scope of the invention to the latter.

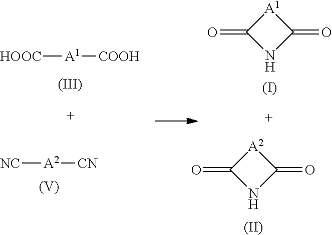

The present invention also aims at the use of cyclic imides of formulae (I) and (II) as defined above, as intermediates for preparing solvents.

The present invention also aims at a process for preparing diester(s) of carboxylic acid(s) which comprises:
the preparation of cyclic imide(s) of formulae (I) and (II) notably according to the process defined above and detailed later on in the description,
the deaminating alcoholysis reaction between the cyclic imides of formulae (I) and (II) and at least one alcohol.

Preparation of the Imides

It will be noted that in the formulae above, if A¹ and A² are identical, the reaction will lead to obtaining only one imide. If A¹ and A² are different, the reaction will lead to obtaining a mixture of imides.

The process for preparing cyclic imide(s) according to the invention may involve at least one carboxylic diacid of formula (III).

According to a first embodiment of the invention, the carboxylic acid of formula (III) may be of biological origin according to the ASTM D6866 standard or obtained by fermentation of sugars, molasses, glucose or starch.

In particular, the carboxylic diacid of biological origin according to the ASTM D6866 standard or obtained by fermentation of sugars, molasses, glucose or starch used in the process of the invention may be selected from succinic acid, glutaric acid, itaconic acid and citraconic acid.

According to a second embodiment of the invention, the carboxylic diacid of formula (III) may be a by-product from the reaction for producing adipic acid. This may be the reaction for producing adipic acid, by nitric oxidation of cyclohexanol or of a cyclohexanol/cyclohexanone mixture. This may also be the reaction for producing adipic acid, by direct oxidation of cyclohexane with a gas containing oxygen, for example oxygen from the air.

In this case, the carboxylic diacid of formula (III) being a by-product from the reaction for producing adipic acid, is a mixture in majority comprising glutaric acid and succinic acid. By <<in majority>>, it is meant that this will be a mixture comprising more than 70% by weight of glutaric acid and of succinic acid. This may notably be a mixture comprising from 40 to 95% by weight of glutaric acid, preferably from 45 to 85% by weight, and from 5 to 60% by weight of succinic acid, preferably from 15 to 55% by weight. Further, the mixture may comprise up to 20% by weight of adipic acid. Such a mixture of carboxylic diacids may be used crude or else purified beforehand, by removal of picric acid, nitric acid, metals and water which it contains. Such a purification treatment may be notably distillation (topping/tailing).

The process for preparing cyclic imide(s) according to the invention may also involve at least one dinitrile of formula (IV).

According to a first embodiment of the invention, the dinitrile of formula (IV) may be of biological origin according to the ASTM D6866 standard. In particular, it may be selected from succinonitrile of biological origin, glutaronitrile of biological origin, itaconitrile of biological origin and citraconitrile of biological origin, all meeting the ASTM D6866 standard.

According to a second embodiment of the invention, the dinitrile of formula (IV) may be a by-product from the reaction for producing adiponitrile, by hydrocyanation of butadiene. In this case when the dinitrile of formula (IV) is a by-product from the reaction for producing adiponitrile by hydrocyanation of butadiene, it is preferably a mixture comprising in majority 2-methylglutaronitrile and 2-ethylsuccinonitrile. By in majority , it will be meant that this is a mixture comprising more than 80% by weight of 2-methylglutaronitrile and of 2-ethylsuccinonitrile. This mixture for example comprises from 70 to 95% by weight of 2-methylglutaronitrile, preferably from 80 to 95% by weight, and from 5 to 30% by weight of 2-ethylsuccinonitrile, preferably from 5 to 20% by weight. Such a mixture of dinitriles may be used crude or else be purified beforehand by removing phosphorus, nitrile pentenes and hydroxylated aromatic compounds which it contains. Such a purification treatment may notably be a topping or an adsorption.

According to a particular embodiment of the invention, the reaction between at least one carboxylic diacid of formula (III) and at least one dinitrile of formula (IV) takes place without any catalyst.

According to another particular embodiment of the invention, the reaction between at least one carboxylic diacid of formula (III) and at least one dinitrile of formula (IV) takes place in the presence of an acid catalyst.

The acid catalyst may be organic or mineral. It is generally a protic acid (Brönsted acid).

The selected acid catalyst will advantageously be an acid which is not very corrosive towards the installations with which it is in contact.

The acid catalyst may be soluble in the mixture of reagents, thereby allowing homogeneous catalysis or else be insoluble in the mixture of reagents, thereby allowing heterogeneous catalysis.

By <<mixture of reagents>>, it will be meant that this is a mixture of carboxylic diacid(s) of formula (III) and of dinitrile(s) of formula (IV) at the beginning of the reaction.

As regards the selection of the acid catalyst soluble in the reaction mixture, it is made from among orthophosphoric acid (H₃PO₄), metaphosphoric, pyrophosphoric, polyphosphoric acid, phosphonic, sulfuric, sulfonic acid and mixtures thereof. Orthophosphoric acid (H₃PO₄) is preferred.

The acid catalyst is preferably used pure or as a concentrated solution, i.e. in a concentration by weight of more than 75%, preferably more than 80%. This may be an aqueous or organic solution, preferably an aqueous solution.

As regards the catalyst insoluble in the reaction mixture, this may be sulfonic resins, preferably NAFION, zeolites, preferably HBEA, HY or HMOR, clay zeolites, preferably montmorillonites, silica-alumina, or alumina zeolites, preferably gamma or alpha alumina. This may also be a phosphoric acid grafted or supported on silica, sulfated or chlorosulfated zirconia, sulfated niobium oxide, sulfated titanium oxide. Preferably, alumina will be selected, preferably gamma alumina. This type of insoluble catalyst is generally used when the reaction is conducted continuously; the catalyst is then implemented in a fixed or fluidized bed, preferably in a fixed bed.

According to a particular embodiment of the process for preparing cyclic imide(s) according to the invention, at least one polymerization inhibitor may be added to the reaction between the carboxylic diacid(s) of formula (III) and the dinitrile(s) of formulae (IV).

The polymerization inhibitor is advantageously selected from diphenols, preferably hydroquinone or 4-ter-butyl catechol and phenothiazines, preferably 8-hydroxyphenothiazine.

The reaction for preparing cyclic imide(s) according to the process of the invention is a stoichiometric reaction involving one mole of carboxylic diacid of formula (III) for one mole of dinitrile of formula (IV). However, the molar ratio between the dinitrile of formula (IV) and the carboxylic diacid of formula (III) is advantageously comprised between 1 and 1.2, limits included. This slight molar excess of dinitrile of formula (IV) may prove to be necessary for compensating the dinitrile losses due to evaporation of said dinitrile during the reaction.

As mentioned earlier, the carboxylic diacid and the dinitrile may be reacted in the presence of an acid catalyst.

The amount of catalyst which is applied in the process of the invention may vary within wide limits. Preferably, the amount of homogeneous acid catalyst represents at most 1% by weight, preferably between 0.01 and 1% by weight, based on the weight of the reaction mixture at the beginning of the reaction.

The temperature at which is applied the reaction for preparing cyclic imides of formulae (I) and (II) depends on the reactivity of the reagents, on their physical properties and on the presence or not of an acid catalyst in the reaction mixture.

Preferably, the reaction is conducted at the reflux temperature of the dinitrile of formula (IV). Generally, this temperature is located between 200° C. and 300° C., preferably between 240° C. and 280° C.

Generally, the reaction is conducted at atmospheric pressure but lower or higher pressures may also be suitable. One operates under autogenous pressure when the reaction temperature is above the boiling temperature of the reagents and/or of the products.

According to a preferred alternative of the process of the invention, the process of the invention is conducted under a controlled atmosphere of inert gases. An atmosphere of rare gases preferably argon may be established, but it is more economical to resort to nitrogen.

The reaction according to the process of the invention is generally accomplished as a bulk reaction, i.e. the reagents are not diluted in a solvent. However, the invention does not exclude the application of a solvent, such as Sulfolane or Isopar.

From a practical point of view, the process may be applied batchwise or continuously.

Practically, the order of application of the reagents for preparing imides is not critical. According to a preferred alternative of the invention, the dinitrile of formula (IV) and then carboxylic diacid of formula (III) and if necessary the soluble acid catalyst are loaded into a stirred reactor.

It is also possible according to a second alternative of the invention to load a homogeneous mixture comprising the dinitrile of formula (IV) and the carboxylic diacid of formula (III) into a reactor in a fixed bed containing the insoluble catalyst.

In the preferred alternative, after putting the reagents into contact, the reaction mixture under stirring is then brought to the desired temperature.

The mixture is left with stirring until complete consumption of the reagents, which may be followed by an analytical method, for example gas chromatography.

In the case of the second alternative, the size of the catalytic bed is adapted to the flow rate of both reagents so as to obtain complete conversion of the lacking reagent at the outlet of the catalytic bed.

At the end of the reaction, and at the temperature of the reaction, a liquid phase is recovered, comprising the imides of formulae (I) and (II). The imides of formula (I) and (II) may then crystallize upon returning to room temperature. This liquid phase at the temperature of the reaction, or the crystals after returning to room temperature, may be directly reused in another process without any purification operation. The latter may also be slightly treated by means of detarring/topping operation(s). Detarring is generally an operation which aims at removing the heaviest products from the degradation of the reagents and products of the reaction. Topping is generally an operation which aims at removing the excess dinitrile.

It is also possible to contemplate at the end of the reaction a separation of the catalyst. If it is insoluble, it may be separated according to a solid/liquid separation technique, preferably by filtration. If the catalyst is soluble, such as for example phosphoric acid, the latter is neutralized by a base such as sodium hydroxide and it is then proceeded with distillation, the neutralized catalyst then being recovered at the foot of the column.

According to the process of the invention, a mixture of imides is obtained, fitting the formulae (I) and (II) mentioned above in the description wherein A¹ and A² have the meanings given earlier.

Preferably, imides of the following formulae, either alone or as a mixture will be preferred:

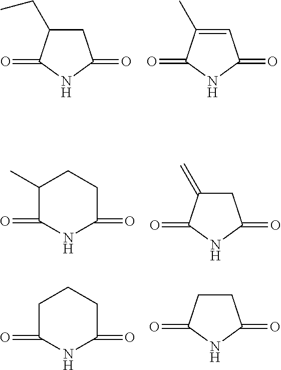

Even more advantageously, the mixtures of cyclic imides comprising:

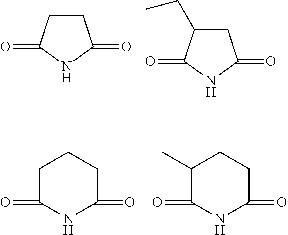

will be preferred.

The process according to the invention described above gives the possibility of obtaining a rate of transformation into dinitrile close to 100% and yields of imides of more than 90%, preferably more than 95%.

The process of the invention is of particular interest since it does not generate any by-product allows total savings of atoms, i.e. the process generates neither nitrogen oxide, nor carbon dioxide, nor ammonia, nor water, nor salt and 100% of the atoms introduced at the beginning of the reaction are again found in the obtained product. It is further simple to apply in industrial and economically performing installations since it is productive. Further it is respectful of the environment since no toxic or harmful product is produced.

The process of the invention also allows the use of bio-sourced reagents, which is particularly advantageous from the industrial point of view considering the decrease in fossil resources.

Further, the process of the invention allows re-upgrading of industrial waste, burnt up to now, into noble products which may be used without any wieldy purification treatment, which represents a significant ecological and economical gain.

The process of the invention also has the advantage of producing imides which may be directly transformed without any additional purification steps, which are generally costly in time and in energy.

The present invention also aims at the use of imides of formula (I) and (II), notably obtained by the process described earlier, as intermediates for preparing solvents. Such solvents may in particular be carboxylic acid diesters.

Preparation of the Diesters

As regards the preparation of diester solvents, the imides of formulae (I) and (II), notably obtained by the process for preparing cyclic imide(s) of formulae (I) and (II) of the invention, may be engaged into a deaminating alcoholysis reaction and lead to the corresponding diesters of carboxylic acids.

This is why the present invention also aims at a process for preparing diester(s) of carboxylic acid(s) which comprises:
the preparation of cyclic imide(s) of formulae (I) and (II) according to the process for preparing cyclic imide(s) of formulae (I) and (II) defined above,
a deaminating alcoholysis reaction between the cyclic imides of formulae (I) and (II) and at least one alcohol.

For the same reasons as those mentioned above for the preparation of the imides, if A¹ and A² are identical, the process above will lead to obtaining a single type of carboxylic acid diester. If A¹ and A² are different, the reaction will lead to obtaining a mixture of two types of carboxylic acid diesters. By <<type of diester>>, it will be understood that the possibility of applying the deaminating alcoholysis reaction in the presence of one or several alcohols may also itself induce the obtaining of a mixture of compounds.

According to an advantageous embodiment of the process for preparing diester(s) of carboxylic acid(s) according to invention, the first step is the preparation of cyclic imide(s) of formulae (I) and (II) according to the process for preparing cyclic imide(s) of formula (I) and (II) above in the description.

In the second step of the process for preparing diester(s) of carboxylic acid(s) according to the invention, the imide(s) of formula (I) and (II) are reacted with at least one alcohol, the alcohol advantageously fitting the following formula (V): R″OH wherein R″ represents a hydrocarbon group comprising from 1 to 20 atoms. The group R″ may be aliphatic, cycloaliphatic, aromatic or arlyalkyl. The group R″ may also comprise heteroatoms or substituants. By <<heteroatom>>, will be meant, as an example and without however being limited thereto, the following atoms: O, N, S, P. By <<substituents>>, will be meant, as an example and without however being limited thereto, the following atoms, Cr, Br, I, F.

Preferably, the alcohol of formula (V) applied in this second step of the process for preparing diester(s) of carboxylic acid(s) according to the invention is selected from the following alcohols: methanol, ethanol, propanol, isopropanol, n-butanol, isobutanol, pentanol, isopentanol, hexanol, cyclohexanol, 2-ethylhexanol, iso-octanol, benzyl alcohol and mixtures thereof.

Mixtures of alcohols may be applied, such as Fusel oil.

According to a particular embodiment of the invention, the reaction between the imides of formulae (I) and (II) and at least one alcohol takes place without any catalyst.

According to a particular embodiment of the invention, the reaction between the imides of formulae (I) and (II) and at least one alcohol takes place in the presence of a catalyst.

The catalyst may be an acid or basic catalyst, preferably an acid catalyst.

By acid catalyst is meant an acid catalyst in the sense of Lewis, as defined in the literature, notably by Jerry MARCH, Advanced Organic Chemistry, 3rd edition, John Wiley and Sons, 1985, pp. 227 and following pages, or a catalyst identified as such in the present application.

The acid catalyst may be soluble in the initial reaction mixture thereby allowing homogeneous catalysis or else insoluble in the initial reaction mixture thereby allowing heterogeneous catalysis. Preferably, it is proceeded with homogeneous catalysis by means of a soluble catalyst in the initial reaction mixture.

By <<initial reaction mixture>> it will be understood that for this deaminating alcoholysis reaction, this is the mixture of imide(s) of formula (I) and (II) and of alcohol(s) at the beginning of the reaction.

As regards the acid catalyst soluble in the initial reaction mixture, this may be a lanthanide salt such as a triflate, chloride or nitrate.

The soluble acid catalyst is preferably used pure or in a concentrated solution, i.e. in a concentration by weight of more than 75%, preferably more than 80%. This may be an alcoholic solution, preferably a methanol solution.

As regards the acid catalyst insoluble in the initial reaction mixture, this may be a solid acid catalyst, typically used in a heterogeneous phase, for example selected from:
metal oxides such as alumina, titanium oxides, silica/alumina mixtures and the like,
zeolites in a acid form,
clays in acid form,
acid phosphates such as NaH₂PO₄ or silicon orthophosphate.

By basic catalyst is meant a basic catalyst in the sense of Lewis, as defined in the literature, notably by Jerry MARCH, Advanced Organic Chemistry, 3rd edition, John Wiley and Sons, 1985, pp. 227 and following pages, or a catalyst identified as such in the present application.

The basic catalyst may be soluble in the initial reaction mixture thereby allowing homogeneous catalysis or else insoluble in the initial reaction mixture, thereby allowing heterogeneous catalysis. By initial reaction mixture it will be understood for this deaminating alcoholysis reaction that this is the mixture of imide(s) of formula (I) and (II) and of alcohol(s) at the beginning of the reaction.

According to a first embodiment, it possible to apply as a soluble basic catalyst, an organic salt comprising a basic anion. Alkaline or earth alkaline salts of compounds comprising a sulfate, sulfonate, phosphate or phosphonate group or of organic compounds comprising a carboxylate or alcoholate (or “alkylate”) group are notably suitable. Mention will notably be made of potassium, sodium or lithium alcoholates, notably sodium ethanolate or lithium ethanolate.

According to a second embodiment, it is possible to apply, as a soluble basic catalyst, a mineral base. This may be a mineral base either containing nitrogen or not.

The mineral bases other than the nitrogen-containing bases have the advantage of being of a more moderate cost and less harmful as regards the environment. Finally, one is protected against any secondary reaction which may be observed with primary or secondary amines for example, notably N-alkylation of the imides formed.

Water-soluble alkaline salts of the hydroxide, inorganic carbonate, inorganic phosphate type are notably suitable. As an illustration of these bases, mention may notably be made of hydroxides such as NaOH, KOH, LiOH and salts of strong bases with a weak acid such as K₂CO₃ and Na₂CO₃, K₃PO₄, Li₃PO₄.

According to a third embodiment, an alkaline metal in the metal form, for example sodium is applied as a soluble catalyst.

According to a fourth embodiment, it is possible to apply as an insoluble basic catalyst, a solid basic catalyst allowing heterogeneous catalysis. In this particular case, the base used may be a heterogeneous catalyst based on hydroxides and/or oxides of alkaline, earth alkaline/or lanthanide elements. This may notably be magnesia (MgO), Mg(OH)₂, CaO, Ca(OH)₂, BaO, Ba(OH)₂, La₂O₃.

In particular, this may be a catalyst selected from earth alkaline and/or rare earth oxides, hydroxides and basic salts not having a valency degree of IV and from minerals containing them.

It is notably possible to apply natural or synthetic analog minerals which consist of intercalated layers based on metal oxides or hydroxides, like hydrotalcite. This may in particular be a natural hydrotalcite or a synthetic analog. These basic salts may contain various combinations of metal cations M²⁺ such as Mg²⁺, Zn²⁺, Ni²⁺, Te²⁺, Co²⁺ and trivalent cations of the M³⁺ type like Al³⁺, Cr³⁺, Fe³⁺. The anions associated with these metal cations may be halogens, organic anions or further oxanions. As an illustration of these hydrotalcites, mention may in particular be made of the one fitting the formula

[Mg₆A₂(O₄)₁₆]CO₃.4H₂O.

It is notably possible to apply oxides and carbonates of rare earths such as ytterbium and lanthanum.

As examples of particularly useful basic catalysts, let us mention:
alcoholates (or “alkylates”), of an alkaline metal, notably sodium methylate, sodium ethylate, sodium tert-butylate, potassium methylate, potassium ethylate, potassium tert-butylate, preferably sodium methylate,
sodium metal,
lanthanum oxide, or
magnesium oxide.

The second step of the preparation of diester(s) of carboxylic acid(s) according to the process of the invention is a reaction involving two moles of alcohol per mole of imide. The reaction step between a carboxylic diacid of formula (III) and a dinitrile of formula (IV) leads to the formation of two moles of cyclic imide(s) of formulae (I) and (II). Thus the molar ratio between the alcohol and the mixture of cyclic imides of formula (I) and (II) is advantageously comprised between 4 and 20, limits included.

The amount of catalyst used during the preparation of diester(s) of carboxylic acid(s) according to the process of the invention is preferably less than 25% by mass, preferably less than 10% by mass, and still more preferentially comprised between 1 and 5% by mass, based on the mass of cyclic imide(s) of formulae (I) and (II).

The temperature at which the deaminating alcoholysis reaction is applied depends on the reactivity of the reagents and on the presence or not of a catalyst in the initial reaction mixture.

The deaminating alcoholysis reaction may be applied in the liquid or vapor phase, preferably in a liquid phase.

Preferably, the deaminating alcoholysis reaction is conducted in the liquid phase at a temperature below 400° C., preferably between 100 and 300° C.

Preferably, the deaminating alcoholysis reaction is conducted at a pressure from 1 to 100 bars, preferably at autogenous pressure.

One operates under autogenous pressure when the reaction temperature is greater than the boiling temperature of the reagents and/or of the products.

During the deaminating alcoholysis reaction, ammonia is formed which is recovered during this step.

The deaminating alcoholysis reaction according to the process of the invention is generally a bulk reaction, i.e. the reagents are not diluted in a solvent, the reagents and notably the alcohol participating in the homogeneity of the initial mixture.

From a practical point of view, the process may be applied batchwise or continuously.

At the end of the deaminating alcoholysis reaction, a liquid phase is recovered, generally after condensation of the gas phase, comprising the diesters of carboxylic acids. The diesters contained in this phase may then be recovered by any conventional means known to one skilled in the art, notably by distillation or extraction.

Separation of the catalyst may also be contemplated at the end of the reaction.

From a practical point of view, the order of application of the reagents for the alcoholic hydrolysis reaction is not critical.

According to a preferred alternative of the invention, the alcohol of formula (V) and then the imides of formulae (I) and (II) and if necessary the catalyst are loaded in a stirred, for example continuously operating, reactor.

It is also possible according to a second alternative of the invention to load a homogeneous mixture comprising the alcohol of formula (V) and the imide(s) of formula (I) and (II) in a fixed bed reactor containing the insoluble catalyst.

After putting the reagents in contact, the reaction mixture with stirring is brought to the desired temperature.

It is left with stirring until complete consumption of the reagents which may be tracked by an analytical method, for example gas chromatography.

According to the process of the invention, a mixture of diester(s) of carboxylic acid(s) fitting the formulae (VI) and (VII) below is obtained, wherein A¹ and A² have the meanings given earlier.

R″OOC-A¹-COOR″ (VI)

and

R″OOC-A²-COOR″ (VII)

Preferably, carboxylic acid diesters will be selected from the following formulae, either alone or as a mixture:

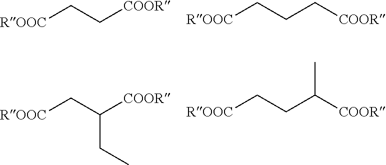

wherein R″ is a methyl, ethyl, propyl, isopropyl, n-butyl, isobutyl, pentyl, isopentyl, hexyl, cyclohexyl, 2-ethylhexyl, iso-octyl, benzyl and mixtures thereof, for example mixtures stemming from Fusel oil. Preferably, R″ is a methyl, ethyl, propyl or mixtures thereof.

Such diesters have diverse applications, notably as solvents in the field of painting, cleaning, stripping and agrochemicals.

Indeed, the diesters of the invention may be used as solvents or co-solvents
in formulations of inks and coatings (paint, varnish, can and coil coating ).
in solutions of resins (phenolic resins for manufacturing foundry molds, etc.)
in formulations for domestic, institutional or industrial cleaning or stripping, notably of resins, inks, paints or graffiti,
in the cleaning and stripping formulations for the electronics industry and photovoltaics,
in lubricant formulations: metalworking, textile, . . .
in pesticide formulations.

Exemplary embodiments of the invention are given hereafter. These examples are given as an illustration and without any limitation.

In the examples, the following abbreviations mean:

The conversion rate (TT) corresponds to the ratio between the number of transformed substrate moles and the number of engaged substrate moles.

The yield (RR) corresponds to the ratio between the number of formed product moulds and the number of engaged substrate moles.

### EXAMPLES

### Example 1

### Preparation of 3-methyl-glutarimide (MGI) Without Any Catalyst

In a 100 mL stirred reactor, are introduced 29.2 g of 2-methyl-glutaric acid and 21.6 g of 2-methyl-glutaronitrile (MGN). With stirring, the reaction mixture is brought to 270° C. and these conditions are maintained for 2 hours. The brown reaction medium is then analyzed by gas chromatography (GC) and the following results are obtained:

TT % (MGN)=99%

RR % (MGI)=97%

### Example 2

### Preparation of 3-methyl-glutarimide (MGI) With an H₃PO₄ Catalyst

In a 100 mL stirred reactor, are introduced 29.2 g of 2-methyl-glutarique and then 21.6 g of 2-methyl-glutaronitrile and 0.05 g of 85% orthophosphoric acid as a catalyst. With stirring, the reaction mixture is brought to 270° C. and these conditions are maintained for 2 hours. The black reaction medium is then analyzed with GC and the following results are obtained:

TT % (MGN)=99%

RR % (MGI)=96%

### Example 3

### Preparation of the Glutaramide Without Any Catalyst

In a 100 mL stirred reactor, are introduced 26.4 g of glutaric acid and 18.8 g of glutaronitrile. With stirring, the reaction mixture is brought to 270° C. and these conditions are maintained for 2 hours. The brown reaction medium is then analyzed with GC and the following results are obtained:

TT % (glutaronitrile)=99%

RR % (glutarimide)=96%

### Example 4

### Preparation of a Mixture of Imides From Pure MGN and From AGS (Glutaric Acid, Succinic Acid and Adipic Acid)

In a 100 mL stirred reactor, are introduced 23 g of 2-methyl-glutaronitrile and then 26 g of a mixture of diacids, by-products of the synthesis of adipic acid, the majority products of which are glutaric acid, succinic acid and a few % by weight of adipic acid. This raw material was treated beforehand for removing picric acid, part of the metals and water. 0.1 g of 85% ortho-phosphoric acid are added and the reaction medium is heated with stirring up to 270° C. After maintaining these conditions for 2 hours, the black reaction medium is analyzed. The following results are obtained:

TT % (MGN)=98%

RR % (MGI)=90%

The formation of other possible imides is observed but the latter are not quantified, (succinimide, glutaramide).

### Example 5

### Preparation of a Mixture of Imides From Pure MGN and From Bio-Sourced Succinic Acid

In a 100 mL reactor, are introduced 23 g of 2-methyl-glutaronitrile and then 25 g of succinic acid obtained by fermentation are added. Stirring is applied and 0.1 g of 85% ortho-phosphoric acid are added. The reaction medium is heated up to 270° C. and these conditions are maintained for 2 hours. By GC analysis, the following results are obtained:

TT % (MGN)=98%

RR % (MGI)=96%

RR % (succinimide)=97%

### Example 6

### Preparation of a Mixture of Diesters

In a 100 mL reactor, are introduced 10 g of a mixture of imides obtained as in Example 5, 100 mL of isobutyl alcohol are added and 2 g of anatase titanium oxide are added. The reaction medium is heated to 250° C., under autogenous pressure. After cooling and filtration of the catalyst, the reaction medium is analyzed by gas chromatography. For a 50% conversion of the sum of the imides, a yield of butyl diesters of 38% is obtained.

### Example 7

### Preparation of a Mixture of Diesters

In a 300 mL reactor, 10 g of a mixture of imides as obtained in Example 4, are introduced, 90 g of methanol and then 1 g of lanthanum oxide (Rhodia) are added. The reaction medium is heated with stirring and under autogenous pressure to 250° C. for 6 hours. After cooling and filtration of the catalyst, the reaction medium is analyzed by gas chromatography. For a 95% conversion of the sum of the imides, a yield of 62% of diesters is obtained.

### Example 8

### Preparation of a Mixture of Diesters

In a 300 mL reactor, are introduced 10 g of a mixture of imides obtained like for example in Example 4. 90 g of methanol and 0.25 g of sodium methylate are added. The reaction medium is heated with stirring under autogenous pressure and these conditions are maintained for 6 hours. After cooling, the reaction medium is analyzed by gas chromatography. For a 92% conversion of the sum of the imides, a yield of a mixture of methyl diesters of 65% is obtained.

1. A process for preparing cyclic imide(s), the process comprising reacting at least one carboxylic diacid of formula (III):
HOOC-A¹-COOH (III),
with at least one dinitrile of the following formula (IV):
NC-A²-CN (IV),
wherein A¹ and A² are identical or different and are selected from the following divalent hydrocarbon groups:
an alkylene comprising a linear linked chain having 2 or 3 carbon atoms;
an ortho-cycloalkylene having 5 or 6 carbon atoms;
an alkenylene comprising a linear link chain having 2 or 3 carbon atoms; and
one or several hydrogen atoms of said hydrocarbon groups optionally substituted with a group R, R being selected from the following substituents: C₁-C₁₀ alkyl, C₅-C₆ cycloalkyl, C₆-C₁₀ aryl, C₆-C₁₀ alkylaryl, C₆-C₁₀ arylalkyl, hydroxy or halogeno group;
one or several hydrogen atoms of the alkylene and cycloalkylene groups optionally substituted with a group R′, R′ being a C₁-C₁₀ alkylidene, wherein the cyclic imide(s) have a structure corresponding to formulae (I) and (II): 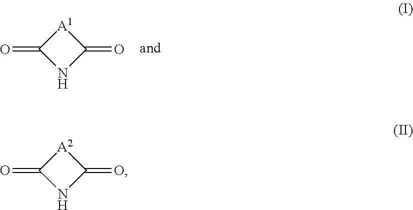
wherein A1 and A2 are defined above.

2. The process as defined by claim 1, wherein the at least one carboxylic diacid of formula (III) is of biological origin according to the ASTM D6866 standard or obtained by fermentation of sugars, molasses, glucose or starch.

3. The process as claim 2, wherein the carboxylic diacid of formula (III) of biological origin according to the ASTM D6866 standard or obtained by fermentation of sugars, molasses, glucose or starch is selected from the group consisting of succinic acid, glutaric acid, itaconic acid and citraconic acid.

4. The process as defined by claim 1, wherein at least one carboxylic diacid of formula (III) is a by-product from a reaction for producing adipic acid by nitric oxidation of cyclohexanol or of a mixture of cyclohexanol and of cyclohexanone.

5. The process as defined by according claim 1, wherein at least one carboxylic diacid of formula (III) is a by-product from a reaction for producing adipic acid by direct oxidation of cyclohexane with a gas containing oxygen.

6. The process as defined by claim 4, wherein the carboxylic diacid of formula (III) is a mixture comprising in majority glutaric acid and succinic acid.

7. The process as defined by claim 1, wherein at least one dinitrile of formula (IV) is of biological origin according to the ASTM D6866 standard.

8. The process as defined by claim 7, wherein the dinitrile of formula (IV) of biological origin according to the ASTM D6866 standard is selected from the group consisting of succinonitrile, glutaronitrile, itaconitrile and citraconitrile.

9. The process as defined by claim 1, wherein at least one dinitrile of formula (IV) is a by-product from a reaction for producing adiponitrile by hydrocyanation of butadiene.

10. The process as defined by claim 9, wherein the dinitrile of formula (IV) is a mixture comprising in majority 2-methylglutaronitrile, and 2-ethylsuccinonitrile.

11. The process as defined by claim 1, wherein the reaction between the at least one carboxylic diacid of formula (III) and the at least one dinitrile of formula (IV) takes place without any catalyst.

12. The process as defined by claim 1, wherein the reaction between the at least one carboxylic diacid of formula (III) and the at least one dinitrile of formula (IV) takes place in the presence of an acid catalyst.

13. The process as defined by claim 12, wherein the acid catalyst is a soluble catalyst in the reaction mixture, allowing homogeneous catalysis.

14. The process as defined by claim 12, wherein the acid catalyst is an insoluble catalyst in the reaction mixture, allowing heterogeneous catalysis.

15. The process as defined by claim 12, wherein the acid catalyst accounts for at most 1% by weight based on the weight of the reaction mixture at the beginning of the reaction.

16. The process as defined by claim 1, wherein the molar ratio between the dinitrile of formula (IV) and the carboxylic diacid of formula (III) is between 1 and 1.2, limits included.

17. The process as defined by claim 1, wherein the reaction is conducted at the reflux temperature of the dinitrile of formula (IV).

18. The process as defined by claim 1, wherein the reaction is conducted at atmospheric pressure.

19. A method of preparing solvents, the method comprising preparing the solvents using of cyclic imides of formulae (I) and (II) obtained by the process as defined by claim 1 as intermediates for preparing solvents.

20. The method as defined by claim 19, wherein the solvents are carboxylic acid diesters.

21. A method for preparing diester(s) of carboxylic acid(s), the method comprising:
preparing cyclic imides of formulae (I) and (II) according to the process as defined by claim 1,
and conducting a deaminating alcoholysis reaction between the cyclic imides of formulae (I) and (II) and at least one alcohol.
